(19) United States
(12) Patent Application Publication
(10) Pub. No.: US 2013/0178731 A1
(43) Pub. Date: Jul. 11, 2013

(54) METHODS AND APPARATUS FOR RISK ASSESSMENT OF DEVELOPMENTAL DISORDERS DURING EARLY COGNITIVE DEVELOPMENT

(71) Applicant: William J. Bosl, Brookline, MA (US)

(72) Inventor: William J. Bosl, Brookline, MA (US)

(73) Assignee: Children's Medical Center Corporation, Boston, MA (US)

(21) Appl. No.: 13/816,645

(22) PCT Filed: Aug. 12, 2011
(86) PCT No.: PCT/US2011/047561

§ 371 Date: Mar. 25, 2013

Related U.S. Application Data
(60) Provisional application No. 61/373,642, filed on Aug. 13, 2010.

(51) Int. Cl. A61B 5/00; A61B 5/04; A61B 5/0476

(52) U.S. Cl. CPC A61B 5/4088; A61B 5/0476; A61B 5/04008; A61B 5/04012; A61B 5/7264
USPC 600/409; 600/300; 600/544

(57) ABSTRACT

The nonlinear complexity of EEG signals is believed to reflect the scale-free architecture of the neural networks in the brain. Analysis of the complexity and synchronization of EEG signals as described herein provides a quantitative measure for routine monitoring of functional brain development in infants and young children and provide a useful biomarker for detecting functional abnormalities in the brain before the cognitive, behavioral or social manifestations of these brain developments can be observed and measured by standard tests. One or more machine learning algorithms are used to discover relevant patterns in the complexity and synchronization values determined from the EEG data to facilitate risk assessment and/or diagnosis of developmental disorders in infants and young children by predicting cognitive, behavioral and social outcomes of the measured functional brain activity patterns.

### BACKGROUND

1. Field of the Invention

This invention relates generally to the analysis of electromagnetic signals to identify biomarkers for cognitive, language and behavioral disorders, of known or unknown etiology (collectively referred to herein as ‘developmental disorders’), and more specifically to analyzing EEG data using complexity and/or synchronization measures in infants to identify characteristics associated with developmental disorders including autism spectrum disorder (ASD).

2. Related Art

Normal and abnormal behavior are differentiated by subtle, complex patterns of activity that an expert clinician observes or discovers through systematic diagnostic tests. In practice, the vast majority of pediatric neuropsychiatric and neurological assessment is based on observing behaviors or by asking caregivers about the child in an effort to understand brain function. Such assessment is particularly difficult in infants and young children who may exhibit a limited set of behaviors and limited communication abilities.

If brain function and behavior are mirrors of each other, as is commonly accepted, then biomarkers of developmental disorders may be hidden in subtle, complex patterns of neurobiological data. Furthermore, the range of mental disorders with a developmental etiology now includes schizophrenia, psychopathy and antisocial behavior disorders, susceptibility to post-traumatic stress syndrome, as well as autism and other pervasive developmental disorders and neurological disorders such as epilepsy that emerge during childhood. An important factor in understanding developmental developmental disorders is the relationship between functional brain connectivity and cognitive, behavioral and language development. This challenge is difficult in part because the brain is a complex, hierarchical system and few methods are available for noninvasive measurements of brain function in developing infants and young children.

The human brain contains on the order of 10¹¹ neurons and more than 10¹⁴ synaptic connections. Although sparsely connected, each neuron is within a few synaptic connections of any other neuron. This remarkable connectivity is achieved by a kind of hierarchical organization that is not fully understood in the brain, but is ubiquitous in nature, called scale-free or complex networks. Complex networks are characterized by dense local connectivity and sparser long-range connectivity that is fractal or self-similar at all scales. A comparison of network properties using functional magnetic resonance imaging (fMRI) showed that children and young-adults' brains have similar “small-world” organization at the global level, but differ significantly in hierarchical organization and interregional connectivity.

The explosive growth of neuroimaging studies that link functional brain activity to behavior promises exciting opportunities for measuring nonlinear brain activity that may indicate abnormalities or allow response to therapy to be monitored. Measurements of brain electrical activity with electroencephalography (EEG) have long been a valuable source of information for neuroscience research, yet this rich resource may be under-utilized for clinical applications in neurology and psychiatry. To fully exploit this data, methods for discovering subtle patterns in nonlinear features and deeper understanding of the relationship between emergent signal features and the underlying neurophysiology are needed.

EEG measurements are safe and the technique is relatively easy to use even with very young children. EEG signals are believed to derive from pyramidal cells aligned in parallel in the cerebral cortex and hippocampus, which act as many interacting nonlinear oscillators. As a consequence of the scale-free network organization of neurons, EEG signals exhibit complex system characteristics reflecting the underlying network topology, including various entropy measures, transient synchronization between frequencies, short and long range correlations and cross-modulation of amplitudes and frequencies. While more research is needed to completely understand the relationship between neural network topology and the characteristics of EEG machine learning algorithms can be used now to find clinically-relevant relationships between signal features and brain function.

Many different methods for computing the complexity of a signal have been defined and used successfully to analyze biological signals. A measure called multiscale entropy (MSE) was shown to be a remarkable biomarker for cardiac health when computed from EKG signals. Sample entropy, upon which MSE is based, has been shown to be significantly higher in certain regions of the right hemisphere in pre-term neonates that received skin-to-skin contact than in those that did not, indicating faster brain maturation. Signal complexity has also been used as a marker of brain maturation in neonates and was found to increase prenatally until maturation at about 42 weeks, then decreased after newborns reached full term. A study of the correlation dimension (another measure of signal complexity) of EEG signals in healthy subjects showed an increase with aging, interpreted as an increase in the number of independent synchronous networks in the brain. Other measures of signal complexity have also been shown to be related to various aspects of brain function and cognition, including the scale dependent Lyapunov exponent (SDLE).

### SUMMARY OF THE INVENTION

The inventor has recognized and appreciated that measureable nonlinear features in electromagnetic EEG signals may potentially be used as biomarkers of normal or abnormal cognitive development. In particular, methods from complex systems theory for analyzing the depth of information contained in these signals may be used to characterize functional brain development during early childhood. To this end, some embodiments are directed to analyzing electromagnetic data using one or more measures of complexity and/or synchronization to characterize developmental disorders such as autism.

Although the techniques described herein are generally applicable to the analysis of electromagnetic data to characterize brain function, some embodiments are particularly directed at analyzing electromagnetic data recorded from infants and young children who, as discussed above, may have limited behavioral and/or communication repertoires. Accordingly, some embodiments are directed to analyzing the complexity and/or synchronization of EEG data collected from infants and/or young children to elucidate brain functions that may not be observable at such a young age. Such quantitative measures of brain function may provide a reliable way to perform risk assessment and/or diagnosis of neurodevelopmental abnormalities early in life.

The neurophysiological mechanisms that underlie normal and abnormal cognitive function may not be understood by pure reduction to physiological causes. The dynamics of the brain are inherently nonlinear, exhibiting emergent dynamics such as chaotic and transiently synchronized behavior that may be central to understanding the mind-brain relationship or the ‘dynamic core’. Some studies suggest that complex mental disorders such as autism cannot easily be described as associated with underconnectivity, but clearly exhibit abnormal connectivity that may vary between different regions. In the autistic brain, high local connectivity and low long-range connectivity may develop concurrently due to problems with synapse pruning or formation. Similarly, neural connectivity patterns that lead to other developmental disorders are not described simply as too many or few neural connections (synapses). Accordingly, some embodiments are directed to estimating changes in neural connectivity in the developing brain using nonlinear techniques as such changes may be used as an effective diagnostic marker for abnormal connectivity development.

A great deal of information about interrelationships in the nervous system likely remains hidden because the linear analysis techniques currently used to analyze neurobiological data fail to detect these interrelationships. Accordingly, some embodiments are directed to using chaotic signal and phase synchronization analyses of electromagnetic data. Such analyses arose from a need to rigorously describe physical phenomena that exhibited what was formerly thought to be purely stochastic behavior but was then discovered to represent complex, aperiodic yet organized behavior, referred to as self-organized dynamics. The analysis of signal complexity and interaction between signals leading to transient synchronization may reveal information about local neural complexity and long-range communication between brain regions, reflecting the underlying neural connectivity structure.

Some embodiments have applications related to methods, computer-readable media, and/or computer systems for risk assessment and/or diagnosis of one or more developmental disorders based, at least in part, on complexity and/or synchronization techniques applied to EEG data collected from infants or young children. The quantities computed from EEG data by these various techniques are collectively referred to as EEG ‘signal features’ or ‘feature set’ or simply ‘features’. For example, some embodiments may be directed to:
Using at least one machine learning algorithm to classify a feature set including EEG measurements collected at multiple time intervals;
Applying at least one nonlinear method of analyzing the complexity and/or synchronization pattern in EEG signals to identify biomarkers of brain development;
Classifying infants into abnormal development or typical development categories using multiscale entropy and phase synchronization determined from EEG measurements;
Predicting scores on standardized tests (e.g., Autism Diagnostic Observation Scale (ADOS), Mullen tests) from nonlinear EEG features using supervised learning algorithms;
Combining entropy and synchronization features identified in EEG data to extract characteristic patterns of developmental disorders including autism;
Determining single growth trajectories or feature vectors for a child by combining nonlinear analyses of EEG data collected at different developmental time points (that is, at different ages, such as 6, 9 and 12 months of age);
Mapping generalized synchronization between EEG channels or signals to characterize abnormal brain connectivity in children at high risk to develop autism;
Assigning risk for at least one developmental disorder based, at least in part, on classifying, with a supervised machine learning algorithm, patterns in an EEG feature vector; and
Monitoring the progress of a therapy provided to children at risk for developing developmental disorders by tracking complexity and/or synchronization measures of EEG data collected at multiple timepoints throughout the therapy.

It should be appreciated that all combinations of the foregoing concepts and additional concepts discussed in greater detail below (provided that such concepts are not mutually inconsistent) are contemplated as being part of the inventive subject matter disclosed herein.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows examples of common time series and the corresponding multiscale entropy curves in accordance with some embodiments;

FIG. 2 is a plot of mean multiscale entropy calculated over all EEG electrodes that shows differences between controls and high-risk children in accordance with some embodiments;

FIG. 3 is a plot showing the scalp distribution of modified sample entropy for different groups of children in accordance with some embodiments;

FIG. 4 is a flow chart of an EEG data collection and processing technique in accordance with some embodiments;

FIG. 5 is a flow chart of a risk classification technique in accordance with some embodiments; and

FIG. 6 is an exemplary computer system on which some embodiments may be implemented.

### DETAILED DESCRIPTION

Following below are more detailed descriptions of various concepts related to, and inventive embodiments of, methods and apparatus according to the present disclosure for analyzing electromagnetic data. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

In some embodiments, electromagnetic data collected from infants or young children may be analyzed using one or more measures of complexity or synchronization. The electromagnetic data may be collected in any suitable way and embodiments are not limited in this respect. Any suitable electromagnetic data may be used in accordance with embodiments including, but not limited to magnetoencephalography (MEG) and EEG.

In one implementation, resting state EEG using a 64 channel Sensor Net System and signals was recorded using Netstation software available from EGI, Inc. Measurements were taken from a total of 143 infants ranging in age from 6 to 18 months. The distribution of infants in each group (HRA: high-risk for autism, CON: typically developing controls) is illustrated in Table 1. The data were amplified, band-pass filtered (0.1 to 100.0 Hz) and sampled at a frequency of 250 Hz.

| | Age (months) | | | | |
|---|---|---|---|---|---|
| | 6 | 9 | 12 | 18 | All |
| # HRA | 19 | 13 | 31 | 12 | 75 |
| # CON | 21 | 13 | 26 | 8 | 68 |
| Total | 40 | 26 | 57 | 20 | 143 |

The collected EEG data may be analyzed using one or more of the entropy, complexity and/or synchronization techniques described herein or any other suitable measure of entropy, complexity and/or synchronization and embodiments are not limited in this respect. It should be appreciated that any EEG data may be used in accordance with embodiments of the invention, including, but not limited to, EEG data that was collected for some purpose other than use with the analysis techniques described herein.

In one embodiment, twenty seconds of continuous EEG data from all channels was used to compute modified sample entropy on multiple scales as follows (See Bosl, et al., 2011 for more details). Multiple scale time series are produced from the original signal using a coarse graining procedure (e.g., see Costa et al. Physical Review, 2005, 71 (2 Pt 1), pp. 021906, the entirety of which is incorporated by reference herein). The scale 1 time series is the original time series. Scale 2 time series is obtained by averaging 2 successive values from the original series. Scale 3 is obtained by averaging every three original values and so on as shown in equation 1.

$$\begin{matrix}\begin{matrix}{s}_{1} \text{:} {x}_{1} , {x}_{2} , {x}_{3} , … , {x}_{N} \\ {s}_{2} \text{:} ( {x}_{1} + {x}_{2} ) / 2 , ( {x}_{1} + {x}_{2} ) / 2 , ( {x}_{3} + {x}_{4} ) / 2 , … , ( {x}_{N - 1} + {x}_{N} ) / 2 \\ ⋮ \\ {s}_{20} \text{:} ( {x}_{1} + … + {x}_{20} ) / 20 , ( {x}_{21} + … {x}_{40} ) / 20 , … , ( {x}_{N - 20} + {x}_{N} ) / 20\end{matrix} & ( 1 )\end{matrix}$$

Coarse grained series up to scale 20 were computed for each of the 64 EEG channels. The modified sample entropy (mSE) defined in Costa et al. was used to compute the entropy of each time series. The mSE algorithm uses a sigmoidal function to compare vector similarity rather than a Heaviside function with a strict cutoff as with the Sample Entropy sometimes used for analysis of biological and EKG signals. A practical effect of using the modified sample entropy is the computed entropy values are more robust to noise and results are more consistent with short time series.

The modified multiscale entropy (mMSE) was computed from the EEGs for all infants using the modified multiscale entropy algorithm described above. In brief, the similarity functions Aᵣ^{m} and Bᵣ^{m} defined by equations (7) and (9) in Costa et al. were computed m=2 and r=0.15 for each coarse-grained time series defined in equation 1 above. The mMSE for scale s with finite length time series is then approximated by:

$$mMSE ( s , m , r ) = - ln ( \frac{{A}_{r}^{m}}{{B}_{r}^{m}} ) \qquad ( 2 )$$

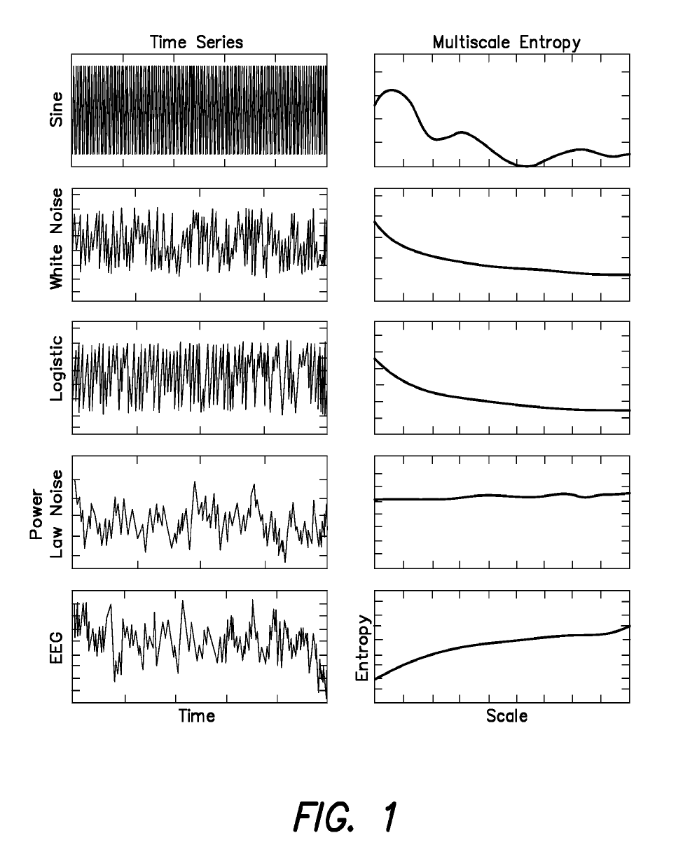

Examples of mMSE curves for several different time series are shown in FIG. 1. Note that white noise and the completely deterministic logistic equation have similar multiscale entropy curves. While the EEG time series shown is visually similar to white noise, its mMSE is quite distinct from all of the other mMSE curves shown. Plots in FIG. 1 where entropy decreases when moving from left to right indicate that a signal contains information only on the smallest scale. In general, if the entropy values across all scales for one time series are higher than for another, then the former is more complex or has greater complexity than the latter.

In order to make some general comparisons of EEG complexity between risk groups and different ages, the mean mMSE was computed as a representative scalar complexity value for each of the 64 channels. Group averages and values for subsets of the 64 EEG channels were computed using equation 2 for infants in the normal control and high risk groups.

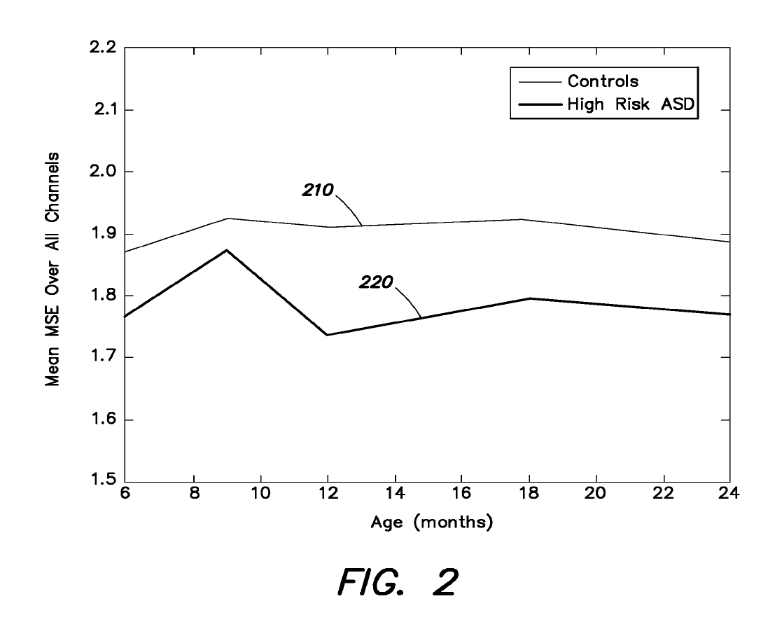

The group average mMSE value versus age for infants in each of the two risk groups is shown in FIG. 2. The bold black line is the mean MSE value averaged over all 64 EEG channels. Left and right laterality were determined by averaging all left-side and all right-side channels separately. Similarly, mMSE values for four left frontal and four right frontal channels were averaged and plotted versus age.

Several features are immediately apparent. A general asymmetry in MSE is apparent in both normal and high-risk groups, although this appears to decline from 12 to 18 months as the left and right hemisphere and frontal curves come closer together at 18 months. EEG complexity changes with age, but not uniformly. In the normal controls, the overall EEG complexity, shown by the solid black line 210, increases from 6 to 9 months, then decreases slightly from 9 to 12 months before increasing again from 12 to 18 months. Left and right channels and the right frontal channels all follow this same pattern, though there is a clear asymmetry between left and right hemisphere complexity. The left frontal channels follow a different pattern, increasing strongly until 12 months, then declining after that. The complexity curve 220 for the high risk group follows a similar pattern, but the overall complexity is lower and the increases and decreases are much more exaggerated. Perhaps even more distinct is the left frontal curve. It follows the same pattern as all other regions but is more accentuated in its decline from 9 to 12 months, unlike the normal controls.

Since the complexity changes seem to vary with EEG channel, a better picture of complexity changes with age and between risk groups may be observed using a scalp plot.

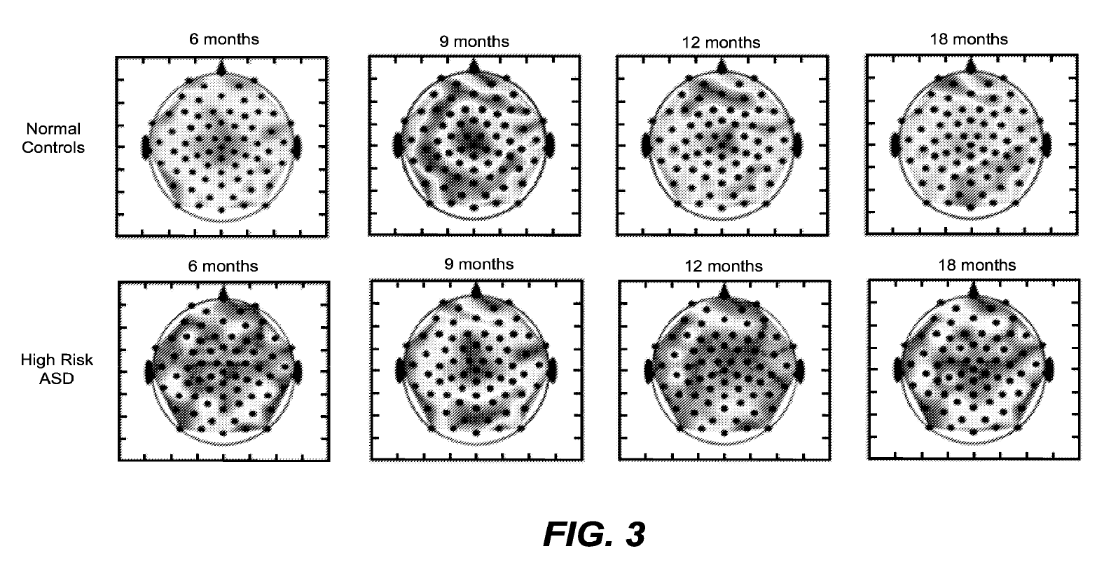

FIG. 3 shows all EEG channels by risk group and age. The complexity values here are computed by averaging the mean mMSE over all coarse grain scales for that channel as in FIG. 2. Complexity variation with age and between risk groups is immediately apparent. One or two channels of the left frontal region appear to increase in complexity continuously with age in the normal controls, as does the right parietal/occipital region. The complexity in the high-risk group is lower than in the control group overall. Although the pattern of complexity change from 6 to 9 months appears similar, the high-risk group shows a marked decline in overall complexity from 9 to 12 months.

Longitudinal studies that compare the MSE trajectories over each brain region may be helpful to determine if characteristic differences can be found that indicate developmental problems. A potential limitation of the data presented herein is that the high-risk group is expected to be quite heterogenous. In the general population, represented by the normal controls, approximately 1 in 150 children are expected to be diagnosed with an ASD after age 3. In the high-risk group, the rate is much higher: 10% to 20% of the infants in this group will later be diagnosed with an ASD. It is not known how many of the high-risk infants exhibit endophenotypes or genetic traits that are indicative of some ASD characteristics, even if they are not later diagnosed with ASD.

The complexity calculations described herein clearly indicate differences between the normal control group and the high-risk group. These complexity differences may reflect endophenotypes (psychiatric biomarkers) that family members may carry even if they do not develop ASD symptoms. Some of the individuals in the high-risk group will develop ASD symptoms of varying severity. The use of EEG signal complexity, as measured by the modified multiscale entropy, may be a sensitive measure of functional brain differences that indicate endophenotypes of ASD or other developmental disorders. As the cohort of children described herein grows older, future EEG measurements, at least through age three years when an official ASD diagnosis can be made, may be informative to compare those in the high-risk group who develop autism from those who do not.

Biological complexity may reflect a systems' ability to quickly adapt and function in a changing environment. The complexity of EEG signals was found in one study to be associated with the ability to attend to a task and adapt to new cognitive tasks; a significant difference in complexity was found between normal subjects and those with diagnosed schizophrenia. Schizophrenic patients were found to have lower complexity than normal controls in some EEG channels and significantly higher interhemispheric and intrahemispheric cross mutual information values (another measure of signal complexity) than the normal controls.

The inventor has recognized and appreciated that other measures in addition to signal complexity may be useful in analyzing electromagnetic data collected from infants and young children. For example, while signal complexity is a property of a single time series or EEG channel, transient synchronized activity is a measure of the interaction between different channels and an indication of communication and coordination between different brain regions. Synchronization may be used as a marker for diagnosing underlying mental disorders such as schizophrenia, autism or epilepsy and may also reveal causal mechanisms. The complexity of synchronization patterns appears to change during network development and reflects different neural wiring schemes and levels of cluster organization.

Additional research is needed to firmly establish the neurophysiological meaning of generalized synchronization between EEG channels. Longitudinal studies to establish baseline synchronization patterns in normal infants at different ages during development and those in people with specific cognitive or mental dysfunctions are needed. A combination of complexity (as measured by, for example, MSE) and generalized synchronization patterns together may give sufficient information about functional brain development to determine if further assessment or early interventions are advised.

However, even if the neurophysiological mechanisms regarding complexity and/or synchronization measures as good biomarkers for mental function or disease are not well understood, the techniques described herein for mapping electrical brain activity, as measured by electromagnetic sensors, to mental and developmental disease using machine learning are nonetheless applicable for assessing risk and/or early diagnosis of developmental disorders. That is, provided that the electromagnetic signals contain diagnostically distinct patterns that are recognized by one or more machine learning algorithms, diagnosis and/or classification in accordance with embodiments is possible, even though the underlying etiology of the electromagnetic signals may be unknown. This is common in medicine: for example, high cholesterol levels were found to be associated with increased risk for heart disease, even before the physiological mechanisms by which high blood cholesterol causes heart attacks were understood.

Patterns of synchronization may be useful as biomarkers for developmental disorders if measured regularly during growth. For example, in normal adults, resting state EEGs contain high mid-range (alpha) frequency activity over occipital regions and low activity in other frequency bands. During childhood and adolescence this pattern is quite different and moves toward adult frequency distributions in a linear trajectory. In one study, EEG coherence at shorter distances in children increased through the teen years while long range synchrony did not vary.

Abnormalities in phase synchronization between multiple bands have been found to be sensitive biomarkers for mental dysfunction in schizophrenic patients. Unfortunately, similar abnormalities in synchronous activity have been found associated with a number of other mental disorders, so further research is required to discover if more refined patterns of synchrony exist for discriminating different disorders or subtypes. A developmental perspective may be useful here. For example, while many attempts to correlate cortical thickness with intelligence have failed, recent research demonstrated that specific characteristic growth trajectories of cortical thickness from infancy to early teen years were highly correlated with above or below average intelligence, suggesting that growth curves of brain function may contain more information than any combination of measurements at one specific age. This may require that routine brain measurements become part of the medical record and algorithms that recognize abnormal trends would need to be used to interpret data after regular cognitive growth checkups.

It should be emphasized that phase synchronization or signal coherence is an inherently nonlinear phenomenon and is not simple correlation. Three different measures of phase synchronization may be distinguished: coupling between brain regions, synchronization across different frequency bands and phase-locking to external stimuli. Research on the neurological and neuropsychological significance of nonlinear synchronization continues and new methods for detecting multichannel, generalized synchronization and clustering for discovery of mutual synchronization in multichannel data continues. To date, application of multichannel clustering and machine learning methods for discovering synchronization patterns have not been applied to EEG data.

Synchronization itself can be manifested in different ways in different systems. The n:m cyclic relative phase index ψ_{1,2} between two signals, φ₁(t) and φ₂ (t), at a specific time t is computed over a time interval using a sliding window as:

ψ_{1,2}^{n,m}(t)=|nφ₁(t)−mφ₂(t)|,mod 1 (3)

where φ(t)=arctan(H(y)/y) and H(y) is the Hilbert transform of the time series y. The mod 1 term ensures that significant phase differences will be detected even in the presence of noise-induced phase jumps. In most cases n=m=1 is assumed, though cross correlation of signals with n!=m is also possible (note: !=means ‘not equal’).

Two signals are defined to be synchronized when ψ_{1,2} is less than a specified constant. The particular algorithm for computing synchronization described in (3) is stable for nonstationary data and will detect synchronization without the need to distinguish between noise and chaos.

In some embodiments, synchronization is determined by computing instantaneous analytic phase and amplitude using Hilbert transforms and search for correlation in each frequency band (6 bands are typically defined for infants) using centered moving averages. This approach finds weak or strong correlations with time lags. For each pair, the relative phase index may be computed and stored in a correlation matrix.

In some embodiments, synchronization is determined using clustering. In this approach, at each time, some channels may be synchronized and it is assumed that bivariate synchronization is transitive; i.e., if A is synced to B and B is synced to C, then A, B and C are considered to be synchronized and form a synchronized cluster, assuming all pairs are above the threshold. A clustering or unsupervised learning algorithm is applied (e.g., Pycluster: http://bonsai.ims.u-tokyo.ac.jp/%7Emdehoon/software/cluster/index.htm) to all channels at a single (averaged) time segment.

In some embodiments, synchronization clusters are compared between different age groups. As the brain develops in infants, cognitive milestones may be accompanied by changes in long-range connectivity, which may be reflected in synchronization patterns, forming clusters of different regions/neuronal ensembles.

Local neural network connectivity undergoes rapid change during early development and this may be reflected in the multiscale complexity and synchronization of EEG signals. Evidence continues to accumulate to support the theory that distant brain regions are integrated into transiently coherent ensembles during information processing tasks. A number of recent studies have demonstrated a link between brain connectivity and complexity or synchronized activity. EEG channel synchronization may provide valuable information about the neural correlates of cognitive processes. Abnormal brain connectivity either locally, regionally, or both may be a root cause of a number of brain disorders and changes in local complexity or synchronous brain oscillations are known to be related to brain connectivity. Early markers for neurological or mental disorders, particularly those with developmental etiologies, may be the growth trajectories of complexity, as measured by MSE and phase or generalized synchronization. More research is needed to determine the underlying physiological causes of the relationship between these measured quantities and cognitive development, though sound theories have been put forth.

The development of novel EEG sensors with improved resolution, together with new source localization algorithms and methods for computing complexity and synchronization in signals promise continued improvement in the ability to measure subtle variations in brain function. Deeper understanding of the relationship between these neurophysiological processes and cognitive function may yield a new window into the mind and provide clinically useful psychiatric biomarkers.

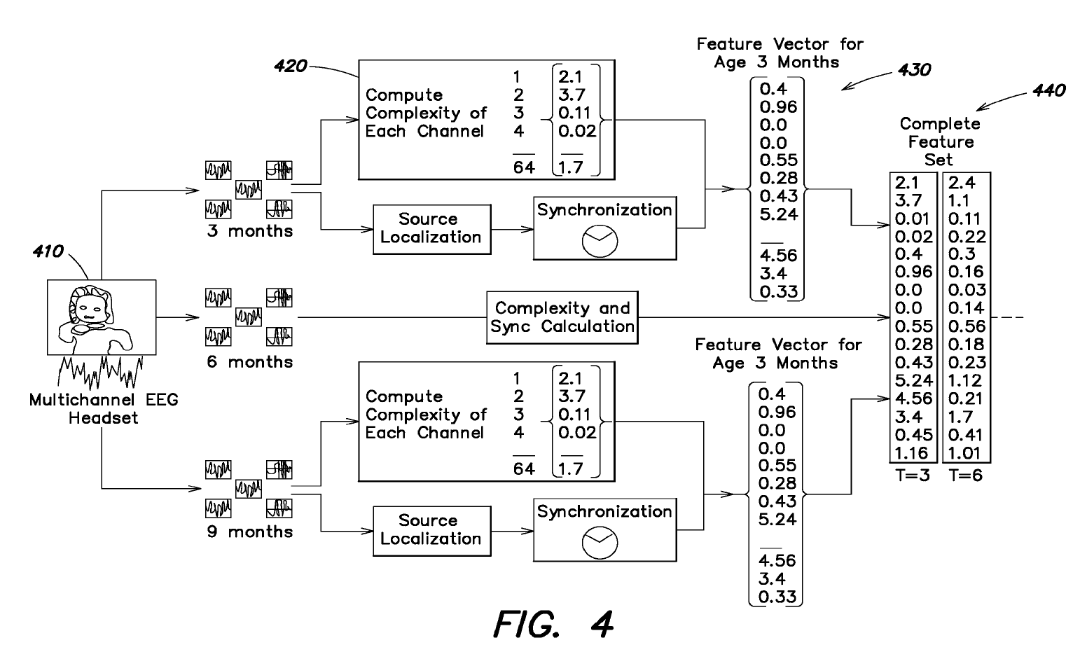

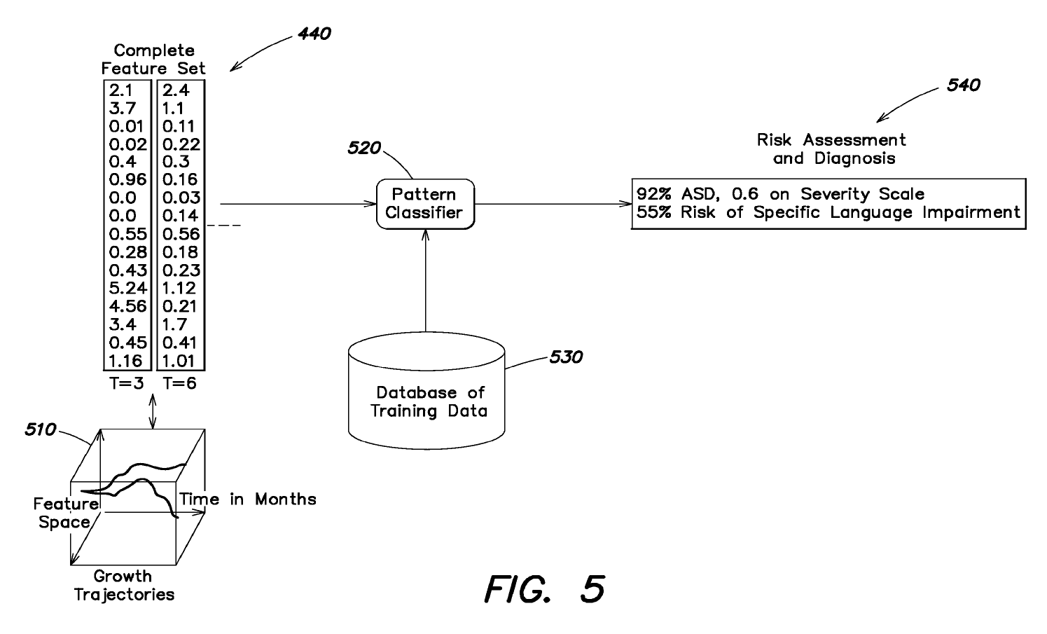

An exemplary flow chart for processing EEG data in accordance with some embodiments is shown in FIGS. 4 and 5. In act 410, EEG data may be collected using a multichannel EEG headset. These measurements may be performed on different groups of children in different age ranges as described above. For example, EEG data may be collected from infants who are three-months old, six-months old, and nine-months old, as shown in FIG. 4 to determine changes in brain function during a development period of interest. Although only three age groups are illustrated in FIG. 4, it should be appreciated that different and/or more age groups may be used with embodiments as the embodiments are not so limited.

After EEG data has been collected, in act 420, complexity and/or synchronization measures based, at least in part, on the EEG data may be determined using one or more techniques described above or other suitable techniques for determining signal complexity or synchronization of electromagnetic signals. The output of the complexity and/or synchronization analyses may be a feature vector 430, which characterizes the EEG measures for a particular child or group of children at a specific age. In some embodiments, EEG recordings 410 and subsequent analysis 420 may be performed at different ages and the feature vectors 430 output from each of the analyses 420 may be combined into a complete feature set 440 for the child over a range of ages, for example 3 to 9 months of age. The complete feature set 440 may then be analyzed using machine learning, a pattern classifier, and/or some other suitable technique for finding patterns in the feature set that have been determined to be associated with autism or other disorder by previous analysis to assess a risk of developing a developmental disorder (e.g., ASD) based on the available EEG data recorded up until the latest measurements. Accordingly, the risk assessment may be continually updated each time new EEG recordings for the child are collected and analyzed in accordance with some embodiments of the invention described herein.

FIG. 5 illustrates a flow chart describing a technique for risk assessment based on a complete feature set 440. After establishing a complete feature set 440 using complexity and/or synchronization analyses performed a multiple time intervals, growth trajectories 510 may be calculated to characterize how components of the complete feature set 510 change over time. In some embodiments, the growth trajectories 510 may be analyzed and classified rather than or in addition to analyzing feature vectors at single age points. For example, the growth trajectories 510 may be used as input data to pattern classifier 520 to predict expert diagnoses, as described in more detail below.

In accordance with some embodiments, the complete feature set 440 may be analyzed using machine learning techniques such as pattern classifier 520 to assess a risk that a child will develop one or more developmental disorders. Pattern classifier 520 receives as input the complete feature set 440 and a database 530 of training data. The database 530 may include any suitable information to facilitate the classification process including, but not limited to known EEG measurements and corresponding expert evaluation and diagnosis. Pattern classifier 520 may implement any suitable machine learning or classification technique including, but not limited to, a support vector machine, k-nearest neighbors, decision tree, a naïve Bayesian algorithm and support vector machine (SVM).

The output of pattern classifier 520 is a risk assessment 540 that details a probability that the child will develop one or more developmental disorders, wherein the probability is based on the complete feature set 440 and the training data stored in database 530, both of which are provided to pattern classifier 520. The ability of pattern classifier 520 to accurately predict a risk assessment 540 may depend on the extent to which pattern classifier 520 is adequately trained with a sufficient amount of known data.

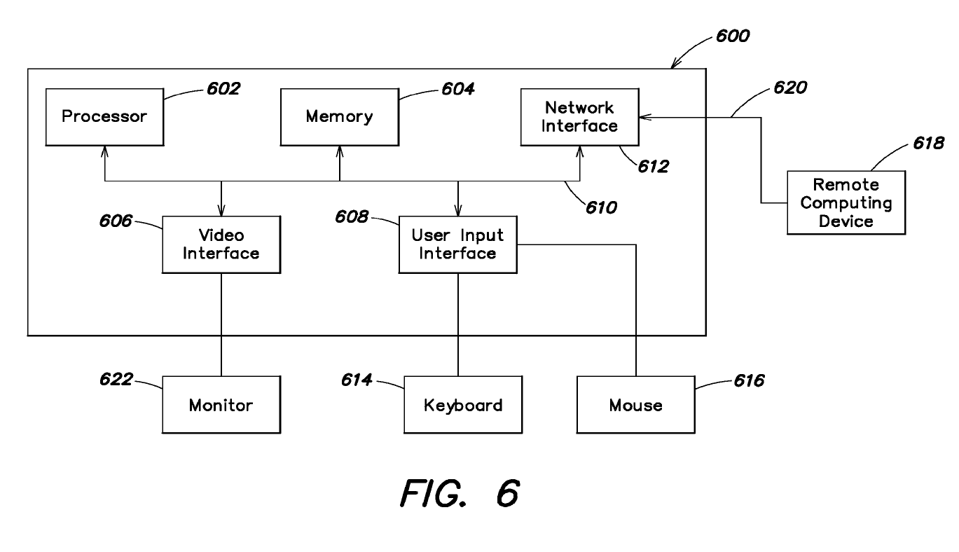

FIG. 6 shows a schematic block diagram of an illustrative computer 600 on which features may be implemented. Only illustrative portions of the computer 600 are identified for purposes of clarity and not to limit aspects of the invention in any way. For example, the computer 600 may include one or more additional volatile or non-volatile memories, one or more additional processors, any other user input devices, and any suitable software or other instructions that may be executed by the computer 600 so as to perform the function described herein. For example, the EEG data may be sent directly from a wireless EEG headset to a smartphone or cell phone and may be relayed directly to the remote computing device 618.

In the illustrative embodiment, the computer 600 includes a system bus 610, to allow communication between a central processing unit 602, a memory 604, a video interface 606, a user input interface 608, and a network interface 612. The network interface 612 may be connected via network connection 620 to at least one remote computing device 618. Peripherals such as a monitor 622, a keyboard 614, and a mouse 616, in addition to other user input/output devices may also be included in the computer system, as the invention is not limited in this respect.

The methods and apparatus disclosed herein may be applied with respect other mental disorders that may have a developmental component in that brain developments or neural correlates emerge in childhood sometimes long before the cognitive, behavioral, or neurological manifestations are observed. Examples of these types of mental disorders that the disclosed methods and apparatus can be applied to include, but are not limited to, schizophrenia, bipolar disorder, susceptibility to post traumatic stress disorder (PTSD), and Alzheimer's disease.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Through, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, embodiments may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect of this embodiment, one or more computer programs that when executed perform methods of embodiments need not reside on a single computer or processor, but may be distributed in a modular fashion amongst a number of different computers or processors to implement various embodiments.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationships between data elements.

Various aspects of embodiments may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, embodiments may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described at least one illustrative embodiment of the invention, various alterations, modifications, and improvements will readily occur to those skilled in the art. Such alterations, modifications, and improvements are intended to be within the spirit and scope of the invention. Accordingly, the foregoing description is by way of example only and is not intended as limiting. The invention is limited only as defined in the following claims and the equivalents thereto.

What is claimed is:

1. A method of analyzing electromagnetic data, the method comprising:
applying, with at least one processor, at least one nonlinear analysis to the electromagnetic data to generate at least one feature set; and
classifying the at least one feature set using at least one machine learning algorithm.

2. The method of claim 1, further comprising:
determining a risk factor for a developmental disorder based, at least in part, on the classified at least one feature set.

3. The method of claim 1, wherein the electromagnetic data comprises electroencephalographic (EEG) data.

4. The method of claim 1, wherein the electromagnetic data comprises magnetoencephalographic (MEG) data.

5. The method of claim 3, further comprising:
collecting the EEG data from a child at different developmental timepoints;
wherein applying the at least one nonlinear analysis comprises applying the at least one nonlinear analysis to the EEG data collected at each developmental timepoint to generate a plurality of feature sets.

6. The method of claim 1, wherein applying the at least one nonlinear analysis comprises applying a complexity analysis.

7. The method of claim 6, wherein the complexity analysis comprises a modified multiscale entropy analysis.

8. The method of claim 7, wherein the electromagnetic data comprises electroencephalographic (EEG) data, the method further comprising:
determining a plurality of scale time series, wherein values in each of the plurality of scale time series are determined by averaging N successive values from the EEG data, where N≧1;
determining an entropy value for each of the plurality of scale time series; and
determining at least one modified multiscale entropy curve based, at least in part, on the entropy values determined for each of the plurality of scale time series.

9. The method of claim 8, wherein determining at least one modified multiscale entropy curve comprises determining a modified multiscale entropy curve for each of a plurality of EEG channel regions.

10. The method of claim 9, wherein the plurality of EEG channel regions include a first region of left hemisphere EEG channels and a second region of right hemisphere EEG channels.

11. The method of claim 6, wherein applying the at least one nonlinear analysis comprises applying the complexity analysis to individual channels of the electromagnetic data to generate a feature set for each channel.

12. The method of claim 11, further comprising:
grouping feature sets for at least some neighboring channels; and
displaying the grouped feature sets as a scalp map.

13. The method of claim 1, wherein applying the at least one nonlinear analysis comprises applying a synchronization analysis, wherein synchronization comprises correlation and coherence.

14. The method of claim 13, wherein the synchronization analysis comprises a generalized synchronization analysis comparing electromagnetic data from a plurality of channels.

15. The method of claim 13, wherein the synchronization analysis comprises a phase synchronization analysis.

16. The method of claim 13, wherein the phase synchronization analysis is configured to evaluate coupling of the electromagnetic data between at least two brain regions.

17. The method of claim 13, wherein the phase synchronization analysis is configured to evaluate synchronization of the electromagnetic data across different frequency bands.

18. The method of claim 13, wherein the synchronization analysis is configured to evaluate phase-locking of the electromagnetic data to at least one external stimulus.

19. The method of claim 13, further comprising:
determining an instantaneous analytic phase and amplitude using Hilbert transforms; and
searching for correlation between electromagnetic data in at least one frequency band using centered moving averages.

20. The method of claim 13, further comprising:
determining, during a predetermined time segment, which channels have synchronized electromagnetic data;
forming at least one synchronization cluster that includes all channels that are determined to have synchronized electromagnetic data.

21. The method of claim 1, wherein applying the at least one nonlinear analysis comprises applying a complexity analysis and a synchronization analysis to the electromagnetic data;
wherein the at least one feature set represents the results from both the complexity analysis and the synchronization analysis.

22. The method of claim 21, wherein classifying the at least one feature set using at least one machine learning algorithm comprises applying a pattern classifier to the at least one feature set.

23. The method of claim 20, further comprising:
receiving a database of training data; and
wherein classifying the at least one feature set comprises classifying the at least one feature set based, at least in part, on the received training data.

24. The method of claim 23, further comprising:
determining a risk assessment for at least one developmental disorder based, at least in part, on the classified at least one feature set.

25. The method of claim 24, wherein the at least one developmental disorder includes autism spectrum disorder.

26. A method of assessing risk for a developmental disorder based on analysis of longitudinally-collected electromagnetic data, the method comprising:
applying, with at least one processor, at least one nonlinear analysis to first electromagnetic data collected at a first time point to generate a first feature set;
applying the at least one nonlinear analysis to second electromagnetic data collected at a second time point to generate a second feature set;
combining the first feature set and the second feature set into a combined feature set;
classifying the combined feature set using a pattern matching algorithm; and
determining a risk for the developmental disorder based, at least in part on the classified combined feature set.

27. The method of claim 26, further comprising:
receiving third or subsequent electromagnetic data collected at a third or more time point (s);
applying the at least one nonlinear analysis to the third or more electromagnetic data to generate a third feature or more set;
updating the combined feature set to include the third feature set;
reclassifying the combined feature set using the pattern matching algorithm; and
updating the risk for the developmental disorder based, at least in part, on the reclassified combined feature set.

28. A computer system, comprising:
a storage device configured to store electromagnetic data collected from at least one patient; and
at least one processor programmed to:
apply at least one nonlinear analysis to the electromagnetic data to generate at least one feature set; and
classify the at least one feature set using at least one machine learning algorithm.

29. The computer system of claim 28, wherein the storage device is further configured to store a database comprising training data;
wherein the at least one processor is further configured classify the at least one feature set based, at least in part, on the training data.

30. The computer system of claim 28, wherein the at least one processor is further programmed to:
determine a risk for a developmental disorder based, at least in part, on the classified at least one feature set.

31. A computer-readable storage medium encoded with a plurality of instructions that, when executed by a computer performs a method comprising:
applying at least one nonlinear analysis to electromagnetic data to generate at least one feature set; and
classifying the at least one feature set using at least one machine learning algorithm.

32. The computer-readable storage medium of claim 31, wherein the at least one machine learning algorithm is a pattern matching algorithm.

33. The computer-readable storage medium of claim 31, wherein classifying the at least one feature set comprises classifying the at least one feature set based, at least in part, on training data.

34. The computer-readable storage medium of claim 31, wherein the method further comprises:
determining a risk for a developmental disorder based, at least in part, on the at least one classified feature set.

35. The computer-readable storage medium of claim 34, wherein the developmental disorder is autism spectrum disorder.

36. The computer-readable storage medium of claim 31, wherein the method further comprises:
determining an estimate for at least one standardized test based, at least in part, on the at least one classified feature set.

37. The computer-readable storage medium of claim 36, wherein that least one standardized test comprises an ADOS or other test specifically used to diagnose autism spectrum disorder.

38. The computer-readable storage medium of claim 36, wherein that at least one standardized test comprises a Mullen test.

39. The computer-readable storage medium of claim 36, wherein that at least one standardized test comprises a standard diagnostic test for a specific developmental disorder.

40. A method of monitoring progress of a therapy provided to a child at risk for developing a developmental disorder, the method comprising:
determining a first complexity and/or synchronization metric for first electromagnetic data collected prior to initiation of the therapy;
determining a second complexity and/or synchronization metric for second electromagnetic data collected after initiation of the therapy; and
comparing the first metric to the second metric to evaluate the efficacy of the therapy provided to the child.
